(19) United States
(12) Patent Application Publication
(10) Pub. No.: US 2011/0288007 A1
(43) Pub. Date: Nov. 24, 2011

(54) BIOCIDAL FUSION PEPTIDE COMPRISING LL-37

(75) Inventors: Marc Alan Fox, Salisbury (GB); Joanne Elizabeth Thwaite, Salisbury (GB); Timothy Philip Atkins, Salisbury (GB); David Okhono Ulaeto, Salisbury (GB)

(73) Assignee: THE SECREATARY OF STATE FOR DEFENCE, Salisbury, Wiltshire (GB)

(21) Appl. No.: 13/131,817

(22) PCT Filed: Nov. 30, 2009
(86) PCT No.: PCT/GB2009/002783

§ 371 Date: May 27, 2011

(30) Foreign Application Priority Data
Nov. 28, 2008 (GB) 0821687.1

(51) Int. Cl. A61K 38/16; C07H 21/00; A61P 31/14; A61P 31/04; A61P 31/10; C07K 19/00; A61P 31/12

(52) U.S. Cl. 514/2.4; 530/324; 530/326; 536/23.4; 514/3.3; 514/3.7

(57) ABSTRACT

The present invention relates to a biocidal fusion peptide comprising: a) a peptide LL-37 or an active fragment or active derivative thereof; b) and a further heterologous bioactive peptide or an active fragment thereof, wherein part or all of the amino acid sequence of the fusion peptide is predicted to form an alpha-helix structure for disruption of a pathogen membrane, compositions such as pharmaceutical compositions comprising the same, methods of preparing the peptide and use of the peptides in treatment, in particular for the treatment of bacterial infection and/or fungal infection and/or viral infection.

The present disclosure relates to fusion peptides, for example with antibacterial and/or anti-fungal and/or anti-viral activity, compositions such as pharmaceutical compositions comprising the same, methods of preparing the peptides, and use of the peptides in treatment, in particular for the treatment of bacterial infection and/or fungal infection and/or viral infection.

Many different families of anti-microbial peptides, classified by their amino acid sequence and secondary structure have been isolated from insects, plants, mammals and microorganisms.

Melittin is a peptide with antibacterial activity isolated from honey bee venom.

Cecropin, cysteine-containing defensin and sapecin, isolated from insects, are examples of antibacterial peptides whose target site is lipid membrane of Gram positive bacteria. Studies have demonstrated that Cecropin B isolated from Bombix mori has biological activity against bacterial species. Further, it was reported that this peptide when translocated into the intercellular spaces in rice transgenic plants was protected from degradation by plant peptidases and confers enhanced resistance to the rice plants against Xanthomonas oryzae pv. oryzae infection.

Attacin, sarcotoxin, deftericin, coleoptericin, apidaecin and abaecin are other antibacterial peptides whose target site is lipid membranes. These peptides conserve G and P domains, and have an influence on the cell differentiation of Gram negative bacteria. In particular, attacin has been also reported to break down outer membrane of the targeted bacteria by inhibiting the synthesis of outer membrane proteins.

Sarcotoxin IA is an antibacterial peptide that is secreted by a meat-fly Sarcophaga peregrina larva in response to a hypodermic injury or bacterial infection. This peptide is highly toxic against a broad spectrum of both Gram-positive and Gram-negative bacteria and lethal to microbes even at nanomolar concentrations

Several antibiotic peptides have been also isolated from amphibia, and many of them belong to the group of amphipathic alpha-helical structure peptides such as magainins, bombinins, bufonins, dermaseptins and defensins.

According to Zasloff (1987), at least five proteins may be isolated from the skin of the African clawed frog (Xenopus laevis). The natural proteins are active against a broad range of microorganisms including bacteria, fungi and protozoans.

WO 03/010191 describes an anti-microbial peptide, isolated from skin of Phyllomedusa hypochondrialis, a kind of frog native to Amazonian, Brazil.

Anti-microbial activity was described in the U.S. Pat. No. 5,643,876 for peptides derived from magainin. These peptides have a molecular weight of about 2500 Da or less, are highly water soluble, amphiphilic and non-hemolytic.

U.S. Pat. No. 5,424,395 discloses a synthetic peptide with a 23 amino acid sequence, derived from magainin II showing anti-microbial activity in plants. U.S. Pat. No. 5,912,231 discloses a compound comprising a magainin I or a magainin II peptide with biological activity, wherein at least one amino acid residue may be substituted with other amino acids residues.

Defensins are relatively small polypeptides of about 3-4 kDa, rich in cystine and arginine. As a class of anti-microbial peptides, defensins have activity against some bacteria, fungi and viruses. The defensins are believed to have molecular conformations stabilized by cysteine bonds, which are essential for biological activity.

U.S. Pat. No. 5,861,378 and U.S. Pat. No. 5,610,139 disclose peptides isolated from horseshoe crab hemocyte, having a similar amino acid sequence to those of defensin and showing strong anti-microbial activities.

U.S. Pat. No. 5,766,624 discloses a method for treatment of microbe infection in mammals using defensins.

U.S. Pat. No. 5,821,224 describes a defensin of 38-42 amino acids, with anti-microbial activity, obtained from bovine neutrophil.

In U.S. Pat. No. 5,798,336 various peptides with anti-microbial activity are described, related to amino acid sequences within Cathepsin G a granule protein with chymotripsin-like activity.

Another type of anti-microbial peptide named buforin was isolated from the stomach tissue of the Asian toad Bufo bufo garagrizans. Two molecules derived from histone H2A were identified, Buforin I and Buforin II which contain 39-amino acids and 21-amino acids respectively. These molecules showed different mechanisms of action. Buforin II had much stronger anti-microbial activity, killing bacteria without lysing cells and presenting high affinity for DNA and RNA.

U.S. Pat. No. 5,877,274 provides a class of cationic peptides referred to as bactolysins, which have anti-microbial activity.

LL-37 is a human (h) cationic peptide derived from the cathelicidin hCAP-18, which is constitutively expressed by neutrophils, lymphocytes, macrophages, and a range of epithelial cells. Expression is significantly up-regulated in the inflamed skin, in skin lesions from patients with psoriasis and in bronchoalveolar lavage fluid from infants with either systemic or pulmonary inflammation. Expression of LL-37 has also been reported in the Langerhans cells of infants with erythema toxicum. In addition to its antimicrobial and antiendotoxic activities, it has been reported to be chemotactic for monocytes, T lymphocytes, neutrophils, and mast cells, and capable of modulating the expression profile of chemokines, chemokine receptors, and additional genes in macrophages and other mammalian cells. Fusions polypeptides of cecropin and melittin were prepared in Bhargava et al Biophys. J. 2004 January 86(1); 329-336 for investigating the mechanisms employed by the peptides.

WO 2006/138276 also described cecropin and melittin hybrids such as CEME and CEMA, with reduced toxicity to host transgenic plants.

WO 90/11771 discloses antibiotic hybrid peptides where the components are selected from cecropin, cecropin A, cecropin B, cecropin D, melittin, magainin or attacin, with anti-malarial and/or anti-bacterial activity.

Whilst a number of peptides are known they have not been developed for use as antimicrobial agents because generally there are one or more disadvantages associated with their activity, for example some are toxic and have haemolytic effects, and others simply do not have the broad spectrum activity or sufficiently strong activity to render them therapeutically useful.

The present disclosure relates to a biocidal fusion peptide comprising:
a) a peptide LL-37 or an active fragment thereof: and
b) a further heterologous bioactive peptide or an active fragment thereof,
   wherein part or all of the amino acid sequence of the fusion peptide is predicted to form an alpha-helix structure for disruption of a pathogen cell membrane.

Whilst the individual component peptides may have antibacterial activity surprisingly the fusion proteins are not simply active against the entities the individual peptides are active against, that is to say, there is a synergistic effect between the two individual component peptides. Rather the fusion peptides of the disclosure seem to be active against entities that corresponding component peptide(s) are not active against. Thus the fusion peptides of the disclosure may be particularly advantageous in the number/variety of organisms/pathogens against which they are active. In the addition to the advantageous anti-bacterial, anti-fungal and/or anti-viral profile the peptides of the disclosure may have lower toxicity, than certain known antibacterial peptides thereby making them more suitable for administration to humans and/or animals.

If the corresponding component peptides are active against the same pathogens as the fusion peptide, then the fusion peptide may in fact exhibit greater/increased activity against said pathogens.

The peptides according to the disclosure are suitable for treatment of humans and/or animals and at the doses administered/employed they are not cytotoxic to cells.

Furthermore the fusions because they are non-naturally occurring may be more resistant to peptidase degradation, which the unfused peptides may be susceptible to.

Whilst cecropin and melittin fusions have been prepared, it does not seem to have been suggested in the art that preparing fusion peptides comprising LL-37 or fragment thereof as described herein would provide peptides with the advantageous properties.

In one embodiment the fusion peptide comprises 2, 3, or 4, such as 2 bioactive peptides.

In one embodiment at least one peptide employed in the fusion is a membrane disrupting peptide.

In one embodiment at least two peptides employed in the fusion are membrane disrupting peptides.

Biocidal in the context of the present disclosure is intended to refer to peptides capable of damaging, killing, destroying or neutralising pathogens, for example micro-organisms (such as bacteria) and/or viruses. Pathogen in the context of the present disclosure does not include parasites, such as malarial parasites.

In one or more embodiments the peptides of the disclosure are bioactive membrane interfering peptides, in particular, prokaryotic cell-membrane interfering peptides.

Bioactive membrane interfering peptides are those which are capable of damaging or destroying the membranes of various pathogens, the pathogens could, be for example, bacteria and/or viruses.

Bioactive prokaryotic cell-membrane interfering peptides are those which are capable of infiltrating, disrupting, pore-forming, thereby by damaging or destroying the membrane of a prokaryotic cell.

Active fragment thereof as employed herein is intended to refer to the activity of the fragment when incorporated into a fusion peptide. It is not necessarily intended to refer to the activity of the unfused fragment.

The targeting of the micro-organisms may be optimized by an overall, net charge on the peptide, such as a positive or negative charge, in particular positive charge. It may be that the net positive charge may assist the peptide targeting the bacteria, which have a net negative charge. In contrast human and animal cells are neutral and thus in this scenario would be less likely to interact with the net charged peptide, thereby making it more likely that the peptide would interact with an oppositely charged mircrobe in the vicinity.

The biocidal peptides of the disclosure are predicted to have an alpha-helix type structure with a hydrophobic loop capable of targeting the lipophillic layer in the target membrane.

Whilst not wishing to be bound by theory it is believed that the ability of the fusion peptide to form an alpha-helix structure in the appropriate environment is crucial the advantageous properties of the peptides.

Most of the peptides without disulfide bridges have random structures in water, and when they bind to a membrane or other hydrophobic environment, or self- aggregate, they form an alpha-helical structure. For example, cecropins and melittin only acquire amphiphilic alpha-helices in membranous environments. It is known that the both dual cationic and hydrophobic nature of the peptides is important for the initial interaction between the peptide and the bacterial membrane.

Software programmes such as SIMPA ('96) can be employed to predict the structure of the peptide from the amino acid structure, thereby allowing suitable/optimized structures to be prepared.

It may be advantageous to group hydrophilic residues such that when a helix is formed they are located/orientated together to form a hydrophilic region. This may reduce the positivity angle of the peptide/helix and ensure efficient docking of the peptide with the target cell membrane. The 2D structure of the helix represented from above may, for example, be referred to by a model known as a Schiffer-Edmonson wheel structure. See for example Biophysical Journal 1967 Vol. 7, page 121 to 135. The structure of the wheel can be used to predict the amino acid grouping, which will be encountered by the target cell membrane. Amino acids may be omitted or substituted to achieve, for example a hydrophilic or hydrophobic section in a part of the molecule, depending on exactly what is required.

In one embodiment the positivity angle is less than 150 degrees.

In one embodiment the positivity angle is about 100 degrees or less.

Whilst not wishing to be bound by theory it is thought that the specific peptides disclosed herein adopt an alpha-helix structure, at least, when in an appropriate membrane environment.

The bioactive heterologous peptides (including active fragments/active domains therefrom) employed are those which are able to adversely affect the normal function of the a microbial or viral cell, for example causing disruptions of the cell membrane, lysis, death prevention of proliferation and/or prevention growth/mitosis or the like.

The bioactive heterologous peptides may, for example be selected from Buforin I, buforin II, bactolysins, attacin, sarcotoxin, deftericin, coleoptericin, apidaecin and abaecin, cecropin, defensin, sapecin, and/or dermaseptins, melittin, magainins (such as magainin II), derived from Xenopus laevis, or derived from Phyllomedusa hypochondrialis and/or LL-37.

In one embodiment the heterologous peptide is cecropin, or an active fragment thereof, for example a fragment KWKLFKKI [Seq ID No: 1].

A fragment of a heterologous peptide is any three or more consecutive amino acids thereof, for example 9 to 15 consecutive amino acids.

The full length sequence for LL-37 is

[Seq ID No: 2]
LLGDFFRKSKEKIGKEFKRIVQRIKDFLRNLVPRTES.

A fragment of LL-37 is any three or more consecutive amino acids thereof, for example 9 to 15 such as 13 consecutive amino acids thereof, in particular FKRIVQRIKD FLR [Sequence ID No 3].

In one embodiment the fusion peptide comprises full length LL-37 or an active fragment thereof, or a corresponding sequence with one amino acid change, which extends to deletion of one amino acid.

In one embodiment the fusion peptide comprises the following sequence FKRIVQRIKD FLR from LL-37.

In one embodiment the LL-37 peptide or fragment thereof is located at the front of the fusion peptide, i.e. residue 1 onwards is derived from LL-37.

In one embodiment the fusion peptide comprises magainin II or an active fragment thereof or a derivative thereof, for example a fragment AK KFAKAFVAEI M [Sequence ID No: 4] and/or GIGKFLH SAKKF [Sequence ID No: 5].

The sequence of wild-type Magainin II is GIGKFLHSAKKFGKAFVGEIMNS [Sequence ID No: 6], which may be employed in the present disclosure.

Alternatively the alaninated derivative with the S⁸, G¹³ and G¹⁸ residues all replaced with As (GIGKFLHAAKKFAKAFVAEIMNS [Sequence ID No: 7]) as reported by Chen et al (1988) FEBS 236 (2), 462-466 may be employed.

Fragments of magainin, for example magainin II include any three or more consecutive amino acids thereof, for example 9 to 15 consecutive amino acids such as 13 consecutive amino acids thereof, in particular AK KFAKAFVAEI M and/or GIGKFLH SAKKF.

In one embodiment the fusion peptide comprises full length or substantially full length magainin (such as magainin II) or a derivative thereof.

Derivatives of magainin II include for example:

(SEQ ID NO: 8)
Gly Ile Gly Lys Phe Leu His Ser Ala Lys Lys Phe
Gly Lys Ala Phe Val Gly Ile Met Lys Ser;
(SEQ ID NO: 9)
Gly Ile Gly Lys Phe Leu His Ser Ala Lys Lys Phe
Gly Lys Ala Phe Val Ala Ile Met Lys Ser;
(SEQ ID NO: 10)
Gly Ile Gly Lys Phe Leu His Ser Ala Lys Lys Phe
Gly Lys Ala Phe Val Phe Ile Met Asn Ser*,
wherein Ser* is D-Serine ;
(SEQ ID NO: 11)
Gly Ile Gly Lys Phe Leu His Ser Ala Lys Lys Phe
Phe Lys Ala Phe Val Phe Ile Met Asn Ser;
(SEQ ID NO: 12)
Gly Ile Gly Lys Phe Leu Lys Ser Ala Lys Lys Phe
Gly Lys Ala Phe Val Phe Ile Met Asn Ser;
(SEQ ID NO: 13)
Gly Ile Gly Lys Phe Leu His Lys Ala Lys Lys Phe
Ala Lys Ala Phe Val Phe Ile Met Asn Ser;
(SEQ ID NO: 14)
Gly Ile Gly Lys Phe Leu Lys Ser Ala Lys Lys Phe
Ala Lys Ala Phe Val Phe Ile Met Asn Ser;
(SEQ ID NO: 15)
Gly Ile Gly Lys Phe Leu His Lys Ala Lys Lys Phe
Ala Lys Ala Phe Val Phe Ile Met Asn Lys;
(SEQ ID NO: 16)
Gly Ile Gly Lys Phe Leu Lys Lys Ala Lys Lys Phe
Gly Lys Ala Phe Val Phe Ile Met Lys Lys;
and
(SEQ ID NO: 17)
Gly Ile Gly Lys Phe Leu His Ser Ala Lys Lys Phe
Gly Lys Ala Phe Val Xaa Ile Met Asn Ser;
wherein Xaa is .epsilon.-Fmoc-lysine.

In one embodiment the peptide comprises magainin (such as magainin II) or an active fragment thereof or an active derivative thereof and LL-37 or an active fragment thereof.

In one embodiment the peptide is as shown in Sequence ID No: 20.

In one embodiment the peptide comprises LL-37 or an active fragment thereof and magainin (such as magainin II) or an active fragment thereof or an active derivative thereof (i.e. where the order is reversed).

In one embodiment the peptide is as shown in Sequence ID No: 19.

In one embodiment the peptide comprises LL-37 or an active fragment thereof and cecropin or an active fragment thereof.

In one embodiment the peptide comprises cecropin or an active fragment thereof and LL-37 or an active fragment thereof.

In one embodiment the peptide is as shown in Sequence ID No: 18.

In one embodiment the peptide is formed such that domains from one peptide form a sandwich around the active domain or full length sequence of another peptide.

In one embodiment the peptide comprises magainin (such as magainin II) or an active fragment thereof or an active derivative thereof and cecropin or an active fragment thereof and LL-37 or an active fragment thereof.

In one embodiment the peptide comprises cecropin or an active fragment thereof and LL-37 or an active fragment thereof and magainin (such as magainin II) or an active fragment thereof or a derivative thereof.

In one embodiment the peptide comprises LL-37 or an active fragment thereof and magainin (such as magainin II) or an active fragment thereof or a derivative thereof and cecropin or an active fragment thereof.

In one embodiment the peptide comprises LL-37 or an active fragment thereof and cecropin or an active fragment thereof and magainin (such as magainin II) or an active fragment thereof or a derivative thereof.

In one embodiment the peptide comprises cecropin or an active fragment thereof and magainin (such as magainin II) or an active fragment thereof or derivative thereof and LL-37 or an active fragment thereof.

In one embodiment the peptide comprises magainin (such as magainin II) or an active fragment thereof or a derivative thereof and LL-37 or an active fragment thereof and cecropin or an active fragment thereof.

Fusion peptide in the context of the present disclosure may include a molecule with 50 amino acids or less, for example 40 or less such as about 10 to 37, particularly 17 to 27, such as 21 to 25.

In one aspect the fusion peptide employed has a weight of 50,000 Daltons or less, such as 40,000, 30,000, or 25,000 Daltons.

The peptides of the disclosure may be effective antibacterial agents, antiviral agents and/or antifungal agents.

Given that the peptides of the disclosure attack the cell membrane of the target entity it is difficult for the target microbes to mutate and become resistant to the polypeptide. This is important because bacteria have been able to mutate to become resistant to many known types of antibiotics.

In one embodiment the fusion peptide comprises the sequence shown in Sequence ID No. 1-18 such as 1, 2, 3, 4, 15, 17 or 18 or a sequence 80, 85, 90, 91, 92, 93, 94, 95, 96, 97, 98 or 99% homologous/identical thereto when analysis is performed against the full length of the sequences being compared.

Analysis for sequence identity/homology may be performed employing software such as BLAST. Degrees of identity can be readily calculated using known computer programs (see Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). For example, simple sequence comparisons can be done on web-sites such as the NCBI website: http://www.ncbi.nlm.nih.gov/BLAST/ (version 2.2.11). As used herein, percentages identity between sequences are measured according to the default BLAST parameters, version 2.2.11. For polypeptides, blastp is used, for example, with the following settings: advanced blasting, low complexity, expect 10, word size 3, blosun 62 matrix, existence: 11, extension: 1 gap costs, inclusion threshold 0.005 and alignment view: hit table. For nucleotide blasting, blastn is used, with low complexity, expect 10, wordsize 11, alignment view: hitable, semi-auto and autoformat.

In another embodiment up to one amino acid in 10 in the sequence is replaced/substituted with an alternative amino acid provided the biological function of the sequence is retained. In one embodiment the replacements are conservative replacements.

In one embodiment a total of 1 to 10, such as 1 to 5, in particular 2, 3 or 4 amino acids are substituted or deleted in comparison to the relevant portion of the original peptide.

The disclosure also extends to pharmaceutical compositions comprising a fusion peptide as defined herein, and a pharmaceutically acceptable excipient such as a diluent or carrier.

In one embodiment the formulation is for topical administration to a wound in the derma or for administration to the lungs. In this embodiment the formulation may be provided as a solution wherein the diluent is, for example saline, sterile water, a dextrose solution or phosphate buffer solution. Liquid formulations may also contain other ingredients for example preservatives, such as benzalkonium chloride, which is commonly used in pharmaceutical compositions.

Formulations for topical administration, including for administration to the lungs, may also be formulated as dry powders, for inhalation or for dusting the wound or infected area. Dry powder formulations may comprise, for example lactose and will need to have a particle size less than 10 microns if the formulations are to be administered to the lungs. Dry powder formulation may be particularly useful in the treatment of fungal infections, such as athletes foot, onychomycosis, tinea unguium, pityriasis versicolor and/or candida albicans.

Suitable formulations wherein the carrier is a liquid (including a solution or suspension) for administration as, for example, nasal spray, nasal drops, or by aerosol administration by nebulizer, may include aqueous or oily solutions of the active ingredient.

Liposome carriers when employed in the formulations of the disclosure may serve to target a particular tissue or infected cells, as well as increase the half-life of the active. Liposomes include emulsions, foams, micelles, insoluble monolayers, liquid crystals, phospholipid dispersions, lamellar layers and the like. Liposomes may be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), U.S. Pat. Nos. 4,235,871, 4,501,728, 4,837,028, and 5,019,369.

The liposomes generally contain a neutral lipid, for example phosphatidylcholine, which is usually non-crystalline at room temperature, for example eggyolk phosphatidylcholine, dioleoyl phosphatidylcholine or dilauryl phosphatidylcholine.

In one embodiment the formulation is provided as a formulation for infusion. The peptide may for example be lyophilised for reconstitution with sterile water or aqueous buffer solution.

The disclosure also extends to use of peptides or compositions comprising the same as defined herein for the treatment or prophylaxis of bacterial and/or viral infections and/or fungal infections (for example as described herein).

The fusion peptides of the disclosure may be particularly useful in the treatment of S aureus and/or B. cepacia and/or Y. pseudotuberculosis infections.

The peptides according to the disclosure also appear to be particularly useful for neutralising B. anthracis spores and/or B. anthracis vegetative and/or B. subtilis spores and/or B. subtilis vegetative. Even more particularly, the peptide according to the disclosure are useful for neturalising B. anthracis Ames and UM23-C12 spores and B. anthracis UM23-C12 and Ames vegetative.

The disclosure also includes methods of treatment or prophylaxis of bacterial and/or viral infections and/or fungal infections comprising administering a therapeutically effective amount of a peptide or composition as described herein.

In one embodiment, the disclosure includes the use of the CaLL peptide (SEQ ID NO: 18) in the manufacture of a medicament against B. anthracis, in particular, B. anthracis spores UM23-c12, B. anthracis vegetative UM23-C12, B. anthracis spores Ames, B. anthracis vegetative Ames, b. anthracis spores STI, B. anthracis exponential STI; Brucella, in particular Brucella melitensis, more particularly Brucella melitensis 16M; Y. pseudotuberculosis, particularly YPIII; VEEV, in particular VEEV TrD, Fe37 and BeAn; and/or S. aureus, particularly S. aureus ATCC29213 infection.

In another embodiment, the disclosure includes the use of the LLaMA peptide (SEQ ID NO: 19) in the manufacture of a medicament against B. anthracis, in particular, B. anthracis spores UM23-c12 and B. anthracis vegetative UM23-C12; B. cepacia, particularly B. cepacia J2540; Y. pseudotuberculosis, particularly YPIII; and/or S. aureus, particularly S. aureus ATCC29214 infection.

In another embodiment, the disclosure includes the use of the MALL peptide (SEQ ID NO: 20) in the manufacture of a medicament against B. anthracis, in particular, B. anthracis spores UM23-C12, B. anthracis spores Ames, B. anthracis vegetative Ames, B. anthracis spores STI, B. anthracis exponential STI; and/or F. tularensis, in particular, F. tularensis LVS infection.

Similarly, in one embodiment, the disclosure includes a method of treating B. anthracis, in particular, B. anthracis spores UM23-c12, B. anthracis vegetative UM23-C 12, B. anthracis spores Ames, B. anthracis vegetative Ames, b. anthracis spores STI, B. anthracis exponential STI; Brucella, in particular Brucella melitensis, more particularly Brucella melitensis 16M; Y. pseudotuberculosis, particularly YPIII; VEEV, in particular VEEV TrD, Fe37 and BeAn; and/or S. aureus, particularly S. aureus ATCC29213 infection using the CaLL peptide (SEQ ID NO:18).

In another embodiment, the disclosure includes a method of treating B. anthracis, in particular, B. anthracis spores UM23-c12 and B. anthracis vegetative UM23-C 12; B. cepacia, particularly B. cepacia J2540; Y. pseudotuberculosis, particularly YPIII; and/or S. aureus, particularly S. aureus ATCC29214 infection using the LLaMA peptide (SEQ ID NO: 19).

In another embodiment, the disclosure includes a method of treating B. anthracis, in particular, B. anthracis spores UM23-C12, B. anthracis spores Ames, B. anthracis vegetative Ames, B. anthracis spores STI, B. anthracis exponential STI; and/or F. tularensis, in particular, F. tularensis LVS infection using the MALL peptide (SEQ ID NO: 20).

Whilst the dose will depend on a number of variables such as the age and weight of the patient and infection being treated, a dose in the range 1 μg to 500 mg per Kg may be suitable, for example 10 μg to 1 mg per Kg.

When administered topically to the skin as a solution formulation a concentration in the range 0.1 to 10% w/w or w/v such as 0.5 to 5% w/w or w/v may be appropriate.

Also encompassed within the scope of the present disclosure is a detergent formulation as an antibacterial and/or antiviral agent for treating surfaces (comprising a peptide as defined herein).

The disclosure also relates to use of peptides as preservatives, for example in food preparation, cosmetics and/or pharmaceutical formulations and products.

The disclosure also relates to polynucleotide sequences such as DNA sequences encoding said peptides.

The disclosure also extends to hosts comprising said encoding polynucleotides.

A method of preparing a peptide recombinantly in a host is also provided.

It is also envisaged that one or more embodiment described herein may be combined, as technically appropriate.

In the context of this specification “comprising” is to be interpreted as “including”.

Aspects of the disclosure comprising certain elements are also intended to extend to alternative embodiments “consisting” or “consisting essentially” of the relevant elements.

### EXAMPLES

### Example 1

Strains were grown to mid-exponential phase in LB broth at 37° C. or used in their sporulated form. Aliquots of these cultures containing approximately 1×10⁶ CFU/ml were separately exposed to PBS (control) or 25 μM of each peptide. Cultures were maintained at 37° C., 180 rpm throughout the assay. Samples were taken at 0, ½, 1, 2, 3 and 4 hours, then serially diluted in PBS and enumerated on LB agar. Viable CFU/ml counts were obtained following incubation at 37° C.

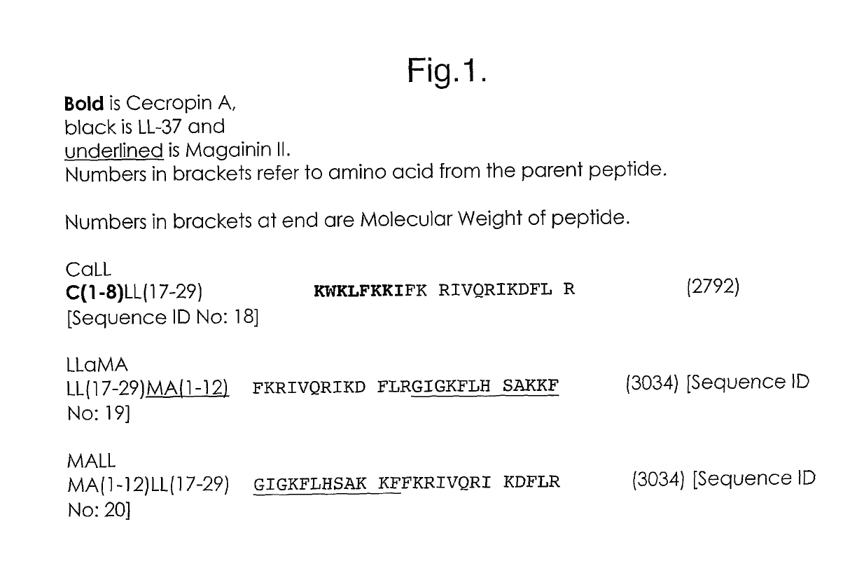

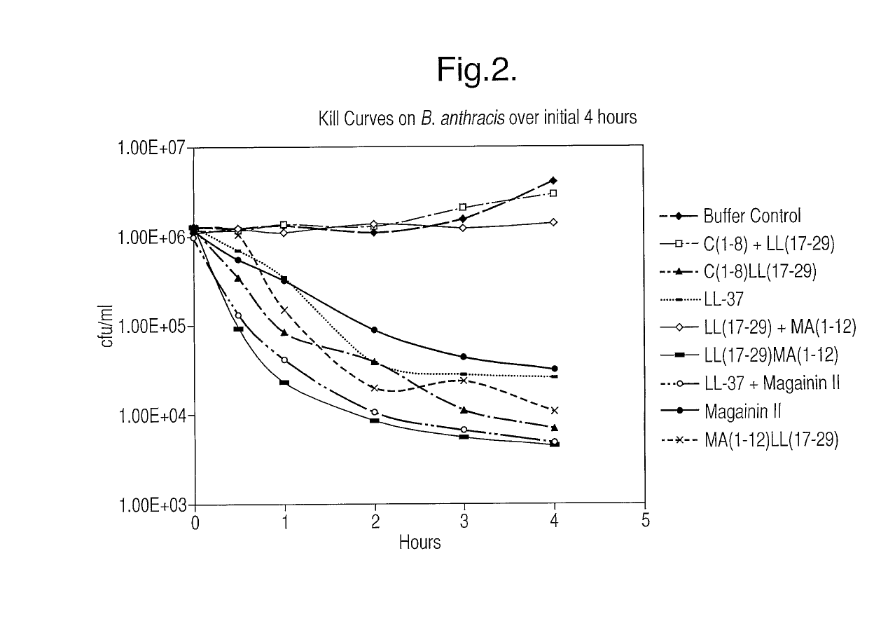

The results for B. anthracis UM23-C12 spores are shown in FIG. 2. It is evident that the buffer control and the combination of unfused peptide fragments (ie C1−8+LL17−29 & LL17−29+MA1−12) had no antibacterial activity. In contrast the fusion peptides in general showed better activity than the natural peptides. The results for B. anthracis Ames spores are shown in FIG. 3.

### Example 2

Table 1 below shows the activity of various peptides against a number of pathogens. Peptides were employed at 102 μg/mL and the data is provided as summaries of the reduction of the number of bacteria present.

### Example 3

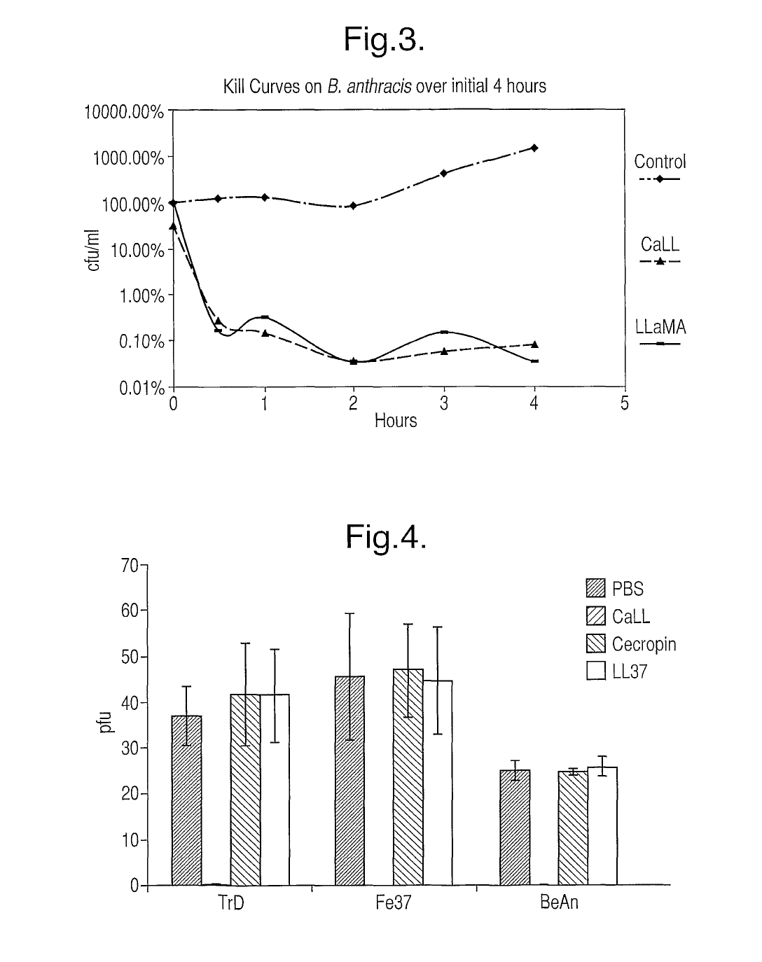

Approximately 100 particles of VEEV (Venezuelan Equine Encephalitis virus) strains TrD, Fe37c or BeAn were incubated with 5 μg of various peptides for 90 mins at 37° C. The number of remaining virus particles was then determined by plaque titration in a sensitive cell line. The results are shown in FIG. 4. It can be seen from the figure that the CaLL hybrid peptide demonstrates activity against several strains of VEEV.

TABLE 1
Effect of peptides after 4 hours incubation with bacteria

| | B. anthracis | B. anthracis | | B. | Y. | Y. | F. | F. | |
|---|---|---|---|---|---|---|---|---|---|
| | Spores | Vegetative | B. cepacia | thailandensis | pseudotuberculosis | pseudotuberculosis | novicida | tularensis | S. aureus |
| | UM23-Cl2 | UM23-Cl2 | J2540 | E264 | IP32593 | YPIII | U112 | LVS | ATCC29213 |
| LL-37 | *** | — | — | — | — | — | — | — | — |
| Magainin II | ** | *** | ** | — | — | *** | — | — | **** |
| CaLL | *** | **** | ** | — | — | “****” | — | — | **** |
| LLaMA | *** | **** | *** | — | — | **** | — | — | **** |
| MALL | *** | * | — | — | — | — | — | *** | — |

| | Bacillus | Bacillus | Bacillus | Bacillus | | |
|---|---|---|---|---|---|---|
| | anthracis | anthracis | anthracis | anthracis | Brucella | Brucella |
| | Spores | vegetative | Spores | exponential | melitensis | suis |
| | Ames | Ames | STI | STI | 16M | 1330 |
| CaLL | *** | **** | *** | **** | *** | * |
| MALL | *** | **** | *** | **** | S | * |

Key
— No effect (i.e. no significant difference between control [culture with no peptide] and culture with peptide)
* ~1 log effect (i.e. culture with peptide that's growth was ~1 log less than control)
** 1-2 log effect
*** 2-4 log effect
**** >4 log effect
“Immediate effect” (i.e. loss of growth by at least 1 log compared to control immediately on addition of peptide [T = 0]

1. A biocidal fusion peptide comprising:
a) a peptide LL-37 or an active fragment or active derivative thereof:
b) a further heterologous bioactive peptide or an active fragment thereof, wherein part or all of the amino acid sequence of the fusion peptide is predicted to form an alpha-helix structure for disruption of a pathogen membrane.

2. The fusion peptide of claim 1 wherein the fusion peptide is predicted to form an alpha-helix structure for disruption of a pathogen cell membrane.

3. The fusion peptide according to claim 1, which comprises a fragment of LL-37.

4. The fusion peptide according to claim 3, wherein the fragment of LL-37 is FKRIVQRIKD FLR.

5. The fusion peptide according to claim 1, which comprises a fragment of magainin II.

6. The fusion peptide according to claim 5, wherein the fragment is selected from AK KFAKAFVAEI M and GIGKFLH SAKKF.

7. A fusion peptide according to claim 1, with an amino acid sequence as defined in Seq ID No 18.

8. A fusion peptide according to claim 1, with an amino acid sequence as defined in Seq ID No: 19.

9. A fusion peptide according to claim 1, with an amino acid sequence as defined in Seq ID No: 20.

10. A fusion peptide according to claims 7 with 80% homology to a sequence defined therein.

11. A pharmaceutical composition comprising a fusion peptide as defined in claim 1.

12. A fusion peptide according to claim 1 for use in treatment.

13. The fusion peptide or composition of claim 12, for the treatment of bacterial infection, viral infection and/or fungal infection.

14. A polynucleotide encoding a peptide as defined in claim 1.

15.-18. (canceled)

19. The fusion peptide of claim 7, for the treatment of viral infection.

20. The fusion peptide of claim 7, for the treatment of VEEV infection.

21. A composition as defined in claim 11 for use in treatment.


SEQUENCE LISTING (20 sequences)

SEQ ID NO: 1 - protein, 8 aa, Hyalophora cecropia
KWKLFKKI

SEQ ID NO: 2 - protein, 37 aa, Homo sapiens
LLGDFFRKSKEKIGKEFKRIVQRIKDFLRNLVPRTES

SEQ ID NO: 3 - protein, 13 aa, Homo sapiens
FKRIVQRIKDFLR

SEQ ID NO: 4 - protein, 13 aa, Artificial sequence
  Synthetic sequence Magainin II derivative
AKKFAKAFVAEIM

SEQ ID NO: 5 - protein, 12 aa, Xenopus laevis
GIGKFLHSAKKF

SEQ ID NO: 6 - protein, 23 aa, Xenopus laevis
GIGKFLHSAKKFGKAFVGEIMNS

SEQ ID NO: 7 - protein, 23 aa, Artificial sequence
  Synthetic sequence Alaninated derivative of magainin II reported by Chen et al. (1988) FEBS 236 (2), 462 - 466
GIGKFLHAAKKFAKAFVAEIMNS

SEQ ID NO: 8 - protein, 22 aa, Artificial sequence
  Synthetic sequence Magainin II derivative
GIGKFLHSAKKFGKAFVGIMKS

SEQ ID NO: 9 - protein, 22 aa, Artificial sequence
  Synthetic sequence Magainin II derivative
GIGKFLHSAKKFGKAFVAIMKS

SEQ ID NO: 10 - protein, 22 aa, Artificial sequence
  Synthetic sequence Magainin II derivative
GIGKFLHSAKKFGKAFVFIMNX

SEQ ID NO: 11 - protein, 22 aa, Artificial sequence
  Synthetic sequence Magainin II derivative
GIGKFLHSAKKFFKAFVFIMNS

SEQ ID NO: 12 - protein, 22 aa, Artificial sequence
  Synthetic sequence Magainin II derivative
GIGKFLKSAKKFGKAFVFIMNS

SEQ ID NO: 13 - protein, 22 aa, Artificial sequence
  Synthetic sequence Magainin II derivative
GIGKFLHKAKKFAKAFVFIMNS

SEQ ID NO: 14 - protein, 22 aa, Artificial sequence
  Synthetic sequence Magainin II derivative
GIGKFLKSAKKFAKAFVFIMNS

SEQ ID NO: 15 - protein, 22 aa, Artificial sequence
  Synthetic sequence Magainin II derivative
GIGKFLHKAKKFAKAFVFIMNK

SEQ ID NO: 16 - protein, 22 aa, Artificial sequence
  Synthetic sequence Magainin II derivative
GIGKFLKKAKKFGKAFVFIMKK

SEQ ID NO: 17 - protein, 22 aa, Artificial sequence
  Synthetic sequence Magainin II derivative
GIGKFLHSAKKFGKAFVXIMNS

SEQ ID NO: 18 - protein, 21 aa, Artificial sequence
  Synthetic sequence Fusion peptide
KWKLFKKIFKRIVQRIKDFLR

SEQ ID NO: 19 - protein, 25 aa, Artificial sequence
  Synthetic sequence Fusion peptide
FKRIVQRIKDFLRGIGKFLHSAKKF

SEQ ID NO: 20 - protein, 25 aa, Artificial sequence
  Synthetic sequence Fusion peptide
GIGKFLHSAKKFFKRIVQRIKDFLR